(19) United States
(12) Patent Application Publication
(10) Pub. No.: US 2008/0081779 A1
(43) Pub. Date: Apr. 3, 2008

(54) Mixtures comprising alpha-ambrinol alkyl ethers and 2 alkoxy-9-methylene-2,6,6-trimethylbicyclo[3.3.1]nonanes as fragrances and flavourings

(75) Inventor: Bernd Holscher, Halle (DE)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: SYMRISE GMBH & CO. KG, Holzminden (DE)

(21) Appl. No.: 11/862,421

(22) Filed: Sep. 27, 2007

(30) Foreign Application Priority Data
Sep. 28, 2006 (DE) 10 2006 046 249.1

(51) Int. Cl. A61K 8/33; A23L 1/22; A61Q 13/00

(52) U.S. Cl. 512/22; 426/534

(57) ABSTRACT

A mixture is described which comprises or consists of
(a) one or more compounds of formula (I): 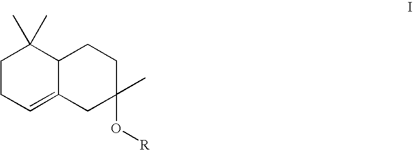 and
(b) one or more compounds of formula (II): 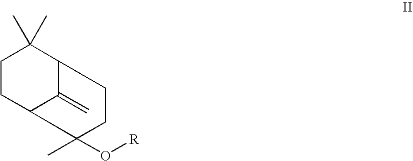 in which R has the same meaning or a different meaning in formula (I) and formula (II) and is selected in each case from the group consisting of methyl and ethyl, and optionally (c) one, two or more other fragrances or flavourings.

### FIELD OF THE INVENTION

The present invention relates primarily to mixtures of specific alpha-ambrinol alkyl ethers and 2-alkoxy-9-methylene-2,6,6-trimethylbicyclo[3.3.1]nonanes which can be used as fragrance and flavouring mixtures. The invention further relates to the corresponding use of a mixture according to the invention as a fragrance or flavouring mixture, to the use of specific 2-alkoxy-9-methylene-2,6,6-trimethylbicyclo[3.3.1]nonanes as agents for masking an unpleasant olfactory note, to the use of said alpha-ambrinol alkyl ethers for modifying the olfactory properties of said 2-alkoxy-9-methylene-2,6,6-trimethylbicyclo[3.3.1]nonanes, to corresponding perfumed products, to corresponding processes for creating, modifying and/or intensifying an odour, and to processes for the preparation of said alpha-ambrinol alkyl ethers and said mixtures. Other features of the present invention are evident from the following description and the attached Claims.

### BACKGROUND OF THE INVENTION

Although a large number of fragrances already exist, there is still a general need for novel fragrances in the perfume industry. Thus there is a need for fragrances with woody, ambrette scents that are capable (in fragrance compositions) of producing not only a woody, ambrette scent but also other interesting olfactory notes, and of broadening the perfumer's possibilities with their novel or original olfactory properties. In particular, there is an interest in fragrances with woody, ambrette scents which are capable of entering into a harmonious combination with flower-scented fragrances. Preferably, the different olfactory aspects and notes should overlap so as to produce an overall complex olfactory impression.

To create novel modern compositions, there is a constant need for fragrances with specific olfactory properties which are suitable for use as a basis for the composition of novel modern perfumes of complex olfactory character. Preferred sought-after fragrances should therefore have not only a woody, ambrette scent but also other notes and aspects which give them olfactory character and complexity.

The search for suitable fragrances and flavourings which led to the present invention was hindered by the following facts:
The mechanisms of odour perception are not adequately known.
The relationships between the special odour perception, on the one hand, and the chemical structure of the corresponding fragrance, on the other, have not been sufficiently researched.
Even slight changes in the structure of a known fragrance often cause substantial changes in the sensory properties and reduce the acceptability to the human organism.

The success of the search for suitable fragrances therefore depends greatly on the intuition of the seeker.

### SUMMARY OF THE INVENTION

The object of the present invention therefore consisted essentially in finding fragrances or flavourings with woody, ambrette scents coupled with other interesting and original olfactory properties, whereby the sought-after fragrances afford novel and original fragrance compositions with specific olfactory notes and aspects. In particular, fragrances with woody, ambrette scents should be found which are especially suitable for combination with fragrances having a flowery scent.

Furthermore, over and above their primary, i.e. olfactory, properties, the fragrances that achieve this main object should also preferably possess additional positive secondary properties, e.g. a higher stability under certain use conditions, a higher strength, a better lingering power, a high substantivity, a noticeable booster effect or a strong blooming, so that sensorially noticeable effects can be achieved, as well as better dermatological and toxicological properties than those of comparable fragrances.

### DETAILED DESCRIPTION OF THE PREFERRED EMBODIMENT

The primary object is achieved according to the invention by the provision of a mixture comprising or consisting of
(a) one or more compounds of formula (I): 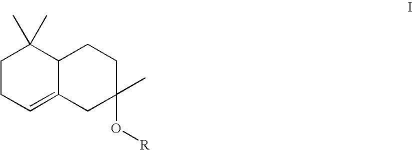
and
(b) one or more compounds of formula (II): 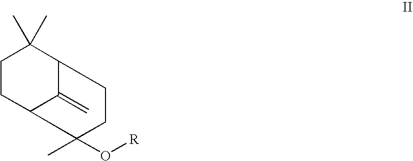
in which
R has the same meaning or a different meaning in formula (I) and formula (II) and is selected in each case from the group consisting of methyl and ethyl,
and optionally
(c) one, two or more other fragrances or flavourings.

The compounds of formula (I) used as component (a) are alpha-ambrinol methyl or ethyl ether, which are formally derived from alpha-ambrinol.

Like alpha-ambrinol, the compounds of formula (I) have two asymmetric carbon atoms. The compounds of formula (I) can exist as an enantiomer of (R,R) configuration, an enantiomer of (R,S) configuration, an enantiomer of (S,R) configuration, an enantiomer of (S,S) configuration or any desired mixture of enantiomers, especially a racemate, or any desired mixture of the corresponding diastereoisomers. If enantiomerically pure or enantiomerically enriched alpha-ambrinol is used as the starting material for the preparation of a compound of formula (I) (see below for preparative process), the particular configurations are retained and enantiomerically pure or enantiomerically enriched alpha-ambrinol alkyl ether is formed. As regards the alpha-ambrinols on which the alpha-ambrinol alkyl ethers are based, reference may be made to Ohloff, G., Fragrance Chemistry, page 546, Academic Press, New York, 1982; P. Naegeli and Y. Wirz-Habersack, Enantiodifferentiation of odour perception of alpha-ambrinols, Tetrahedron: Asymmetry, volume 3, issue 2, 1992, pages 221-222.

The compounds of formula (II) used as component (b) are 2-alkoxy-9-methylene-2,6,6-trimethylbicyclo[3.3.1]nonanes (alkoxy being methoxy or ethoxy).

If one, two or more other fragrances are present as component (c), one, two or more of these perfumes preferably have a woody and/or flowery scent.

The mixtures according to the invention have a surprisingly complex woody, ambrette note; cf. the detailed odour descriptions below. The mixtures according to the invention are therefore outstandingly suitable for use as fragrance or flavouring mixtures.

Over and above their primary, i.e. olfactory, properties, the mixtures according to the invention additionally have positive secondary properties. Particularly when used under acidic conditions, the mixtures according to the invention have a considerable stability advantage over fragrances with similar olfactory properties.

Helv. Chim. Acta 1955, 38, 1573 reports the cyclization of alpha-dihydroionone to a compound of formula (II) above in which R=ethyl. The odour of this compound—corresponding to that of the unetherified alcohols—is described as warmly woody-ambrette, sometimes with a more or less pronounced note of camphor.

European patent specification EP 0 330 995 B1 describes compounds of formula (II) above which exist as mixtures of corresponding exo and endo forms. It is stated that these mixtures of the exo and endo forms are distinguished by powerful and very natural-warm notes with nuances of tobacco, ambergris and wood. The exo isomer is described as sensorially more interesting. According to EP 0 330 995 B1, the ethyl ether (compound of formula (II) above in which R=ethyl) is of central interest and has a woody, amber-like, flowery odour (with a nuance of iris root) with aspects of patchouli, muscatel sage and tobacco. The methyl ether is said to have similar olfactory properties.

Patent specification U.S. Pat. No. 2,803,662 likewise describes compounds of formula (II) above.

The compound of formula (II) in which R=ethyl, i.e. the compound 2-ethoxy-9-methylene-2,6,6-trimethylbicyclo[3.3.1]nonane, has the CAS number 68845-00-1 and is commercially available.

The publication WO 82/00030 (Willis et al.) describes, inter alia, the preparation of alpha-ambrinol methyl ether (i.e. the compound of formula (I) above in which R=methyl) via the etherification of alpha-ambrinol with methyl iodide. However, a sensory description of the ether was not given.

Our own investigations showed that the compounds of formula (I), i.e. alpha-ambrinol methyl ether and alpha-ambrinol ethyl ether, have an unpleasant odour; this odour initially seemed to rule out use in fragrance mixtures. The odour descriptions are as follows:
1. alpha-ambrinol methyl ether (compound of formula (I) in which R=methyl): strongly animal, faecal, indole/skatole, similar to alpha-ambrinol;
2. alpha-ambrinol ethyl ether (compound of formula (I) in which R=ethyl): animal, phenolic, reminiscent of styrax.

Surprisingly, however, despite the animal or animal-faecal olfactory notes of the alpha-ambrinol alkyl ethers, the mixtures according to the invention are outstandingly suitable for use as fragrances. In fact, our own investigations revealed two unexpected sensory effects that seem to be based on a currently inexplicable interaction between the compounds of formula (I) and the compounds of formula (II):

On the one hand, surprisingly, the compounds of formula (II) are capable of masking unpleasant olfactory notes otherwise caused e.g. by the compounds of formula (I). In particular, the strongly animal and faecal olfactory notes of alpha-ambrinol methyl ether and the animal olfactory note of alpha-ambrinol ethyl ether are masked by the compounds of formula (II). The resulting mixture according to the invention thus has none of the animal or faecal olfactory notes produced by the ambrinol ethers or, if only a small amount of compounds of formula (II) is used in comparison with the amount of compounds of formula (I) present, said mixture has only a residual animal or faecal olfactory note whose strength is less than would actually have been expected on the basis of the proportion of compounds of formula (I) in the mixture.

On the other hand, the compounds of formula (I) are capable of modifying the olfactory properties of compounds of formula (II) (with the same or different meanings of R) to make them more radiant with a stronger nuance of ambrette.

The overall impression is that, in mixtures of the compounds of formula (I) and the compounds of formula (II), the unpleasant animal-faecal olfactory impressions which are otherwise created by the alpha-ambrinol alkyl ethers of formula (I) disappear and, instead, the olfactory impression of the compounds of formula (II) becomes more radiant with a stronger nuance of ambrette. The present invention further relates to corresponding uses.

Thus, in the mixtures according to the invention, the compounds of formula (I) do not have the effect that might be expected on the basis of the corresponding odour descriptions of the pure substances. Also, the compounds of formula (II) perform better than those skilled in the art would have expected in view of the state of the art, since they act not only as fragrances but also as masking agents.

Over and above their primary, i.e. olfactory, properties, the mixtures according to the invention comprising compounds of formulae (I) and (II) have additional positive secondary properties. Particularly when used under acidic conditions, the mixtures according to the invention have a considerably higher stability than e.g. alpha-ambrinol.

Preferred mixtures according to the invention comprise or consist of
one compound of formula (I) and one compound of formula (II) with the same meaning of R
   and either
another compound of formula (I) with the other meaning of R and/or
another compound of formula (II) with the other meaning of R
   or
no other compound of formula (I) or (II).

Preferred mixtures according to the invention thus comprise or consist of e.g. a compound of formula (I) in which R is methyl and a compound of formula (II) in which R is methyl, or a compound of formula (I) in which R is ethyl and a compound of formula (II) in which R is ethyl. Preferably, however, R is methyl in both formula (I) and formula (II).

In particular, the mixtures according to the invention which comprise alpha-ambrinol methyl ether (compound of formula (I) in which R=methyl) and 2-methoxy-9-methylene-2,6,6-trimethylbicyclo[3.3.1]nonane (compound of formula (II) in which R=methyl) create a very complex and varied olfactory and gustatory impression which otherwise can only be achieved by complex mixtures of several components (e.g. by ethereal oils or mixtures of herbs or spices).

It is preferable to use pure exo isomers of formula (II) or exo/endo mixtures with a greater proportion of the exo form.

Provided that, in a specific case, there is no wish to have a certain animal or even faecal olfactory note, the total amount of compounds of formula (II) in a mixture according to the invention is chosen so that an animal olfactory note otherwise caused by the compounds of formula (I) is effectively masked.

Preferably, the ratio of the total amount of compounds of formula (I) to the total amount of compounds of formula (II) in a mixture according to the invention ranges from 100:1 to 1:10, preferably from 3:1 to 1:3 and particularly preferably from 2:1 to 1:1.

Particularly preferred compounds according to the invention are shown in the Table below.

| | Ethers of formula II | |
|---|---|---|
| Ethers of formula I | in wt. % | Number of C |
| in wt. % | exo/endo ca. 68:32 | atoms in R |
| 50% | 50% | 1 |
| 50% | 50% | 2 |
| 55% | 45% | 1 |
| 55% | 45% | 2 |
| 75% | 25% | 1 |
| 75% | 25% | 2 |
| 25% | 75% | 1 |
| 25% | 75% | 2 |

The olfactory properties of the preferred mixtures according to the invention given in Table 1 are described as follows: very strongly radiant, ambrette, exalting, soft-woody, reminiscent of ambergris and civet, very natural olfactory impression. The mixtures according to the invention are thus novel and valuable fragrance mixtures whose original olfactory properties are distinctly different from, and superior to, those of known fragrances.

If less than 25 wt. % of 2-methoxy-9-methylene-2,6,6-trimethylbicyclo[3.3.1]nonane (compound of formula (II) in which R=methyl) is used in a mixture that otherwise only comprises alpha-ambrinol methyl ether (compound of formula (I) in which R=methyl), the unpleasant animal-faecal olfactory properties of the compound of formula (I) are no longer completely absent, although this may be desirable in a specific case. If at least 25 wt. % of 2-methoxy-9-methylene-2,6,6-trimethylbicyclo[3.3.1]nonane is present, the unpleasant animal-faecal olfactory properties are absent and the mixture has a lighter, very strongly radiant, more ambrette-like, exalting and soft-woody odour for the perfumer and creates a very natural olfactory impression reminiscent of ambergris and civet. If the proportion of 2-methoxy-9-methylene-2,6,6-trimethylbicyclo[3.3.1]nonane exceeds 75 wt. %, a mixture according to the invention then has a weaker odour again and, as the proportion of the compound of formula (II) increases, no longer achieves a very strongly radiant, ambrette, exalting olfactory impression.

It is understood that preferred mixtures according to the invention have one, several or all of the following olfactory properties: very strongly radiant, ambrette, exalting, soft-woody, reminiscent of ambergris and civet, very natural olfactory impression.

The present invention further relates to the use of a mixture according to the invention as described above (especially in an embodiment previously referred to as preferred) as a fragrance or flavouring mixture, especially as a fragrance having one, several or all of the following olfactory properties: very strongly radiant, ambrette, exalting, soft-woody, reminiscent of ambergris and civet, very natural olfactory impression.

According to a related feature, the present invention further relates to a process for creating, modifying and/or intensifying an odour, comprising the following step:

bringing a product into contact, or mixing it, with a sensorially effective amount of a mixture according to the invention.

Preferably, within the framework of the use according to the invention or the process according to the invention, a mixture according to the invention is used in which the total amount of compounds of formula (II) is chosen so that an animal olfactory note otherwise caused by the compounds (I) is effectively masked. Reference may be made to the details given above as regards the preferred proportions required to achieve this.

The mixtures according to the invention conventionally comprise a sensorially effective total amount of the compounds of formulae (I) and (II). One, two or more other fragrances or flavourings are often present in a mixture according to the invention in addition to the compounds of formulae (I) and (II). The weight ratio of the total amount of compounds of formulae (I) and (II) to be used according to the invention, to the total amount of other fragrances or flavourings, is preferably in the range from 1:1000 to 1:0.50.

Particularly preferably, a mixture according to the invention comprises, as component (c), one, two or more other woody fragrances (incl. sandalwood, musk and amber) selected from the group consisting of sandranol (2-ethyl-4-(2,2,3)-trimethylcyclopent-3-ylbut-2-en-1-ol), Ysamber K (1′,1′,5′,5′-tetramethylhexahydrospiro[1,3-dioxolane-2,8′(5′H)-2H-2,4-a-methanonaphthalene]), globanone (cyclohexadec-8-en-1-one), timberol (1-(2,2,6-trimethylcyclohexyl)hexan-3-ol), iso E Super (2,3,8,8-tetramethyl-1,2,3,4,5,6,8-octahydro-2-naphthalenyl methyl ketone), Cashmeran (1,1,2,3,3-pentamethyl-TH-indan-4-one/6,7-dihydro-1,1,2,3,3-pentamethyl-4(5H)-indanone), iso-bornyl acetate (2-exo-bornanyl acetate) and ylanate (2-tert-butylcyclohexyl acetate).

The combination of a mixture of the compounds of formulae (I) and (II) to be used according to the invention with said woody fragrances produces lighter, more radiant and cleaner olfactory impressions. The corresponding mixtures according to the invention have a more natural and fresher effect.

Another particularly preferred mixture according to the invention comprises one, two or more flowery fragrances selected from the group consisting of lilial (2-methyl-3-(4-tert-butylphenyl)propanal), hedione (methyl (3-oxo-2-pentylcyclopentyl)acetate), mayol (4-isopropylcyclohexylmethanol), linalool (3,7-dimethyl-1,6-octadien-3-ol), dihydromyrcenol (2,6-dimethyl-7-octen-2-ol), citronellol (3,7-dimethyl-6-octen-1-ol), phenoxanol (3-methyl-5-phenylpentanol), 2-phenylethyl alcohol, hydroxycitronellal (3,7-dimethyl-7-hydroxyoctan-1-al) and alpha-ionone (4-(2,6,6-trimethylcyclohex-2-enyl)but-3-en-2-one).

The combination of compounds of formulae (I) and (II) to be used according to the invention with above-mentioned flowery fragrances results in a modification towards freshness and emanation. In addition, the flowery aspects become more rounded. The mixtures have a more intense and more harmonious odour.

The presence of at least one woody fragrance from the above-mentioned group and at least one flowery fragrance from the above-mentioned group is also preferred.

Apart from said combinations with woody and/or flowery fragrances, a combination of the compounds of formulae (I) and (II) to be used according to the invention with green-fruity fragrances is frequently particularly preferred.

Examples of suitable green-fruity fragrances are vertral (octahydro-1H-4,7-methanoindene-5-carbaldehyde), cis-3-hexen-1-ol, beta-damascone (1-(2,6,6-trimethylcyclohex-2-enyl)buten-1-one), vertocitral (2,4-dimethylcyclohex-3-ene-1-carbaldehyde), cyclogalbanate (allyl cyclohexyloxyacetate) and hexyl acetate.

Combination with said green-fruity fragrances produces a rounder and softer odour. Moreover, the addition of the compounds of formulae (I) and (II) to be used according to the invention to the green-fruity fragrances imparts a certain radiance and creates an impression of natural emanation.

It is frequently also advantageous to combine the compounds of formulae (I) and (II) to be used according to the invention with spicy-balsamic fragrances, the following being particularly suitable: eugenol (2-methoxy-4-allylphenol), coumarin (2H-1-benzopyran-2-one), anisaldehyde (4-methoxybenzaldehyde), amylcinnamaldehyde (2-pentyl-3-phenyl-2-propenal), isoamyl salicylate (salicylic acid 3-methylbutyl ester) and cinnamyl alcohol (3-phenyl-2-propen-1-ol).

Aspects of freshness and naturalness are observable when combining the compounds of formulae (I) and (II) to be used according to the invention with said spicy-balsamic fragrances. The mixtures have a more harmonious and more radiant effect.

Quite generally, the compounds of formula (I) and compounds of formula (II) to be used according to the invention can advantageously be combined with a large number of fragrances to give novel fragrance or flavouring compositions. Fragrances (as component (c) of a mixture according to the invention) which are advantageously suitable for combination can be found e.g. in S. Arctander, Perfume and Flavor Materials, vol. I and II, Montclair, N.J., 1969, Eigenverlag, or K. Bauer et al., Common Fragrance and Flavor Materials, 4th edition, Wiley-VCH, Weinheim 2001. The following may be specifically mentioned:

extracts of natural raw materials, such as ethereal oils, concrete oils, absolute essences, resins, resinoids, balsams, tinctures, e.g.:

amber tincture; amyris oil; angelica seed oil; angelica root oil; anise oil; baldrian oil; basil oil; tree moss absolute; bay oil; mugwort oil; benzoin resin; bergamot oil; beeswax absolute; birch tar oil; bitter almond oil; savory oil; buchu leaf oil; cabreuva oil; cade oil; calmus oil; camphor oil; cananga oil; cardamom oil; cascarilla oil; cassia oil; cassia absolute; castoreum absolute; cedar leaf oil; cedarwood oil; cistus oil; citronella oil; lemon oil; copaiva balsam; copaiva balsam oil; coriander oil; costus root oil; cumin oil; cypress oil; davana oil; dill oil; dill seed oil; eau de brouts absolute; oak moss absolute; elemi oil; tarragon oil; eucalyptus citriodora oil; eucalyptus oil; fennel oil; pine needle oil; galbanum oil; galbanum resin; geranium oil; grapefruit oil; guaiacum wood oil; gurjun balsam; gurjun balsam oil; helichrysum absolute; helichrysum oil; ginger oil; iris root absolute; iris root oil; jasmine absolute; calamus oil; blue camomile oil; Roman camomile oil; carrot seed oil; cascarilla oil; Scotch fir needle oil; spearmint oil; caraway oil; ladanum oil; ladanum absolute; ladanum resin; lavandin absolute; lavandin oil; lavender absolute; lavender oil; lemongrass oil; lovage oil; distilled lime oil; pressed lime oil; linaloa oil; litsea cubeba oil; laurel leaf oil; mace oil; marjoram oil; mandarin oil; massoi bark oil; mimosa absolute; ambrette oil; musk tincture; muscatel sage oil; nutmeg oil; myrrh absolute; myrrh oil; myrtle oil; clove leaf oil; clove blossom oil; neroli oil; frankincense absolute; frankincense oil; opopanax oil; orange blossom absolute; orange oil; oreganum oil; palmarosa oil; patchouli oil; perilla oil; Peruvian balsam oil; parsley leaf oil; parsley seed oil; petitgrain oil; peppermint oil; pepper oil; pimento oil; pine oil (Pinus pinea); European pennyroyal oil; rose absolute; rosewood oil; rose oil; rosemary oil; Dalmatian sage oil; Spanish sage oil; sandalwood oil; celery seed oil; lavender spike oil; Japanese anise oil; styrax oil; tagetes oil; fir needle oil; tea tree oil; turpentine oil; thymian oil; tolu balsam; tonka absolute; tuberose absolute; vanilla extract; violet leaf absolute; verbena oil; vetiver oil; juniper oil; wine yeast oil; wormwood oil; wintergreen oil; ylang oil; hyssop oil; civet absolute; cinnamon leaf oil; cinnamon bark oil; and fractions thereof or ingredients isolated therefrom;

individual fragrances from the following groups:

hydrocarbons, e.g. 3-carene; α-pinene; β-pinene; α-terpinene; γ-terpinene; p-cymene; bisabolene; camphene; caryophyllene; cedrene; farnesene; limonene; longifolene; myrcene; ocimene; valencene; (E,Z)-1,3,5-undecatriene; styrene; diphenylmethane;

aliphatic alcohols, e.g. hexanol; octanol; 3-octanol; 2,6-dimethylheptanol; 2-methyl-2-heptanol; 2-methyl-2-octanol; (E)-2-hexenol; 1-octen-3-ol; mixture of 3,4,5,6,6-pentamethyl-3/4-hepten-2-ol and 3,5,6,6-tetramethyl-4-methyleneheptan-2-ol; (E,Z)-2,6-nonadienol; 3,7-dimethyl-7-methoxyoctan-2-ol; 9-decenol; 10-undecenol; 4-methyl-3-decen-5-ol;

aliphatic aldehydes and their acetals, e.g. hexanal; heptanal; octanal; nonanal; decanal; undecanal; dodecanal; 2-methyloctanal; 2-methylnonanal; (E)-2-hexenal; (Z)-4-heptenal; 2,6-dimethyl-5-heptenal; 10-undecenal; (E)-4-decenal; 2-dodecenal; 2,6,10-trimethyl-9-undecenal; 2,6,10-trimethyl-5,9-undecadienal; heptanal diethyl acetal; 1,1-dimethoxy-2,2,5-trimethyl-4-hexene; citronellyloxyacetaldehyde; 1-(1-methoxypropoxy)-(E/Z)-3-hexene;

aliphatic ketones and their oximes, e.g. 2-heptanone; 2-octanone; 3-octanone; 2-nonanone; 5-methyl-3-heptanone; 5-methyl-3-heptanone oxime; 2,4,4,7-tetramethyl-6-octen-3-one; 6-methyl-5-hepten-2-one;

aliphatic sulfur-containing compounds, e.g. 3-methylthiohexanol; 3-methylthiohexyl acetate; 3-mercaptohexanol; 3-mercaptohexyl acetate; 3-mercaptohexyl butyrate; 3-acetylthiohexyl acetate; 1-menthene-8-thiol;

aliphatic nitriles, e.g. 2-nonenoic acid nitrile; 2-undecenoic acid nitrile; 2-tridecenoic acid nitrile; 3,12-tridecadienoic acid nitrile; 3,7-dimethyl-2,6-octadienoic acid nitrile; 3,7-dimethyl-6-octenoic acid nitrile;

aliphatic carboxylic acid esters, e.g. (E)- and (Z)-3-hexenyl formate; ethyl acetoacetate; isoamyl acetate; 3,5,5-trimethylhexyl acetate; 3-methyl-2-butenyl acetate; (E)-2-hexenyl acetate; (E)- and (Z)-3-hexenyl acetate; octyl acetate; 3-octyl acetate; 1-octen-3-yl acetate; ethyl butyrate; butyl butyrate; isoamyl butyrate; hexyl butyrate; (E)- and (Z)-3-hexenyl isobutyrate; hexyl crotonate; ethyl isovalerate; ethyl 2-methylpentanoate; ethyl hexanoate; allyl hexanoate; ethyl heptanoate; allyl heptanoate; ethyl octanoate; ethyl (E,Z)-2,4-decadienoate; methyl 2-octynate; methyl 2-nonynate; allyl 2-isoamyloxyacetate; methyl 3,7-dimethyl-2,6-octadienoate; 4-methyl-2-pentyl crotonate;

acyclic terpene alcohols, e.g. geraniol; nerol; lavadulol; nerolidol; farnesol; tetrahydrolinalool; tetrahydrogeraniol; 2,6-dimethyl-7-octen-2-ol; 2,6-dimethyloctan-2-ol; 2-methyl-6-methylen-7-octen-2-ol; 2,6-dimethyl-5,7-octadien-2-ol 2,6-dimethyl-3,5-octadien-2-ol; 3,7-dimethyl-4,6-octadien-3-ol; 3,7-dimethyl-1,5,7-octatrien-3-ol; 2,6-dimethyl-2,5,7-octatrien-1-ol; and their formates, acetates, propionates, isobutyrates, butyrates, isovalerates, pentanoates, hexanoates, crotonates, tiglates and 3-methyl-2-butenoates;

acyclic terpene aldehydes and ketones, e.g. citronellal; 7-methoxy-3,7-dimethyloctanal; 2,6,10-trimethyl-9-undecenal; geranylacetone; and the dimethyl and diethyl acetals of geranial and neral;

cyclic terpene alcohols, e.g. menthol; isopulegol; alpha-terpineol; terpinen-4-ol; menthan-8-ol; menthan-1-ol; menthan-7-ol; borneol; isoborneol; linalool oxide; nopol; cedrol; ambrinol; vetiverol; guaiol; and their formates, acetates, propionates, isobutyrates, butyrates, isovalerates, pentanoates, hexanoates, crotonates, tiglates and 3-methyl-2-butenoates;

cyclic terpene aldehydes and ketones, e.g. menthone; isomenthone; 8-mercaptomenthan-3-one; carvone; camphor; fenchone; alpha-ionone; beta-ionone; alpha-n-methylionone; beta-n-methylionone; alpha-isomethylionone; beta-isomethylionone; alpha-irone; beta-damascenone; 1-(2,4,4-trimethyl-2-cyclohexen-1-yl)-2-buten-1-one; 1,3,4,6,7,8a-hexahydro-1,1,5,5-tetramethyl-2H-2,4a-methanonaphthalen-8(5H)-one; 2-methyl-4-(2,6,6-trimethyl-1-cyclohexen-1-yl)-2-butenal; nootkatone; dihydronootkatone; 4,6,8-megastigmatrien-3-one; alpha-sinensal; beta-sinensal; acetylated cedarwood oil (methyl cedryl ketone);

cyclic alcohols, e.g. 4-tert-butylcyclohexanol; 3,3,5-trimethylcyclohexanol; 3-isocamphylcyclohexanol; 2,6,9-trimethyl-Z2,Z5,E9-cyclododecatrien-1-ol; 2-isobutyl-4-methyltetrahydro-2H-pyran-4-ol;

cycloaliphatic alcohols, e.g. alpha-3,3-trimethylcyclohexylmethanol; 1-(4-isopropylcyclohexyl)ethanol; 2-methyl-4-(2,2,3-trimethyl-3-cyclopent-1-yl)butanol; 2-methyl-4-(2,2,3-trimethyl-3-cyclopent-1-yl)-2-buten-1-ol; 3-methyl-5-(2,2,3-trimethyl-3-cyclopent-1-yl)-pentan-2-ol; 3-methyl-5-(2,2,3-trimethyl-3-cyclopent-1-yl)-4-penten-2-ol; 3,3-dimethyl-5-(2,2,3-trimethyl-3-cyclopent-1-yl)-4-penten-2-ol; 1-(2,2,6-trimethylcyclohexyl)pentan-3-ol; 1-(2,2,6-trimethylcyclohexyl)hexan-3-ol;

cyclic and cycloaliphatic ethers, e.g. cineol; cedryl methyl ether; cyclododecyl methyl ether; 1,1-dimethoxycyclododecane; (ethoxymethoxy)cyclododecane; alpha-cedrene epoxide; 3a,6,6,9a-tetramethyldodecahydronaphtho[2,1-b]furan; 3a-ethyl-6,6,9a-trimethyldodecahydronaphtho[2,1-b]furan; 1,5,9-trimethyl-13-oxabicyclo[10.1.0]trideca-4,8-diene; rose oxide; 2-(2,4-dimethyl-3-cyclohexen-1-yl)-5-methyl-5-(1-methylpropyl) -1,3-dioxane;

cyclic and macrocyclic ketones, e.g. 4-tert-butylcyclohexanone; 2,2,5-trimethyl-5-pentylcyclopentanone; 2-heptylcyclopentanone; 2-pentylcyclopentanone; 2-hydroxy-3-methyl-2-cyclopenten-1-one; 3-methyl-cis-2-penten-1-yl-2-cyclopenten-1-one; 3-methyl-2-pentyl-2-cyclopenten-1-one; 3-methyl-4-cyclopentadecenone; 3-methyl-5-cyclopentadecenone; 3-methylcyclopentadecanone; 4-(1-ethoxyvinyl)-3,3,5,5-tetramethylcyclohexanone; 4-tert-pentylcyclohexanone; 5-cyclohexadecen-1-one; 6,7-dihydro-1,1,2,3,3-pentamethyl-4(5H)-indanone; 8-cyclohexadecen-1-one; 9-cycloheptadecen-1-one; cyclopentadecanone; cyclohexadecanone;

cycloaliphatic aldehydes, e.g. 2-methyl-4-(2,2,6-trimethylcyclohexen-1-yl)-2-butenal; 4-(4-hydroxy-4-methylpentyl)-3-cyclohexenecarbaldehyde; 4-(4-methyl-3-penten-1-yl)-3-cyclohexenecarbaldehyde;

cycloaliphatic ketones, e.g. 1-(3,3-dimethylcyclohexyl)-4-penten-1-one; 2,2-dimethyl-1-(2,4-dimethyl-3-cyclohexen-1-yl)-1-propanone; 1-(5,5-dimethyl-1-cyclohexen-1-yl)-4-penten-1-one; 2,3,8,8-tetramethyl-1,2,3,4,5,6,7,8-octahydro-2-naphthalenyl methyl ketone; methyl 2,6,10-trimethyl-2,5,9-cyclododecatrienyl ketone; tert-butyl 2,4-dimethyl-3-cyclohexen-1-yl ketone;

esters of cyclic alcohols, e.g. 2-tert-butylcyclohexyl acetate; 4-tert-butylcyclohexyl acetate; 2-tert-pentylcyclohexyl acetate; 4-tert-pentylcyclohexyl acetate; 3,3,5-trimethylcyclohexyl acetate; decahydro-2-naphthyl acetate; 2-cyclopentylcyclopentyl crotonate; 3-pentyltetrahydro-2H-pyran-4-yl acetate; decahydro-2,5,5,8a-tetramethyl-2-naphthyl acetate; 4,7-methano-3a,4,5,6,7,7a-hexahydro-5- or -6-indenyl acetate; 4,7-methano-3a,4,5,6,7,7a-hexahydro-5- or -6-indenyl propionate; 4,7-methano-3a,4,5,6,7,7a-hexahydro-5- or -6-indenyl isobutyrate; 4,7-methanooctahydro-5- or -6-indenyl acetate;

esters of cycloaliphatic alcohols, e.g. 1-cyclohexylethyl crotonate;

esters of cycloaliphatic carboxylic acids, e.g. allyl 3-cyclohexylpropionate; allyl cyclohexyloxyacetate; cis- and trans-methyl dihydrojasmonate; cis- and trans-methyl jasmonate; methyl 2-hexyl-3-oxocyclopentanecarboxylate; ethyl 2-ethyl-6,6-dimethyl-2-cyclohexenecarboxylate; ethyl 2,3,6,6-tetramethyl-2-cyclohexenecarboxylate; ethyl 2-methyl-1,3-dioxolan-2-acetate;

araliphatic alcohols, e.g. benzyl alcohol; 1-phenylethyl alcohol; 3-phenylpropanol; 2-phenylpropanol; 2-phenoxyethanol; 2,2-dimethyl-3-phenylpropanol; 2,2-dimethyl-3-(3-methylphenyl)propanol; 1,1-dimethyl-2-phenylethyl alcohol; 1,1-dimethyl-3-phenylpropanol; 1-ethyl-1-methyl-3-phenylpropanol; 2-methyl-5-phenylpentanol; 3-methyl-5-phenylpentanol; 3-phenyl-2-propen-1-ol; 4-methoxybenzyl alcohol; 1-(4-isopropylphenyl)ethanol;

esters of araliphatic alcohols and aliphatic carboxylic acids, e.g. benzyl acetate; benzyl propionate; benzyl isobutyrate; benzyl isovalerate; 2-phenylethyl acetate; 2-phenylethyl propionate; 2-phenylethyl isobutyrate; 2-phenylethyl isovalerate; 1-phenylethyl acetate; alpha-trichloromethylbenzyl acetate; alpha,alpha-dimethylphenylethyl acetate; alpha,alpha-dimethylphenylethyl butyrate; cinnamyl acetate; 2-phenoxyethyl isobutyrate; 4-methoxybenzyl acetate;

araliphatic ethers, e.g. 2-phenylethyl methyl ether; 2-phenylethyl isoamyl ether; 2-phenylethyl 1-ethoxyethyl ether; phenylacetaldehyde dimethyl acetal; phenylacetaldehyde diethyl acetal; hydratropaldehyde dimethyl acetal; phenylacetaldehyde glyceryl acetal; 2,4,6-trimethyl-4-phenyl-1,3-dioxane; 4,4a,5,9b-tetrahydroindeno[1,2-d]-m-dioxin; 4,4a,5,9b-tetrahydro-2,4-dimethylindeno[1,2-d]-m-dioxin;

aromatic and araliphatic aldehydes, e.g. benzaldehyde; phenylacetaldehyde; 3-phenylpropanal; hydratropaldehyde; 4-methylbenzaldehyde; 4-methylphenylacetaldehyde; 3-(4-ethylphenyl)-2,2-dimethylpropanal; 2-methyl-3-(4-isopropylphenyl)propanal; 2-methyl-3-(4-isobutylphenyl)propanal; 3-(4-tert-butylphenyl)propanal; cinnamaldehyde; alpha-butylcinnamaldehyde; alpha-hexylcinnamaldehyde; 3-methyl-5-phenylpentanal; 4-methoxybenzaldehyde; 4-hydroxy-3-methoxybenzaldehyde; 4-hydroxy-3-ethoxybenzaldehyde; 3,4-methylenedioxybenzaldehyde; 3,4-dimethoxybenzaldehyde; 2-methyl-3-(4-methoxyphenyl)propanal; 2-methyl-3-(4-methylenedioxyphenyl)propanal;

aromatic and araliphatic ketones, e.g. acetophenone; 4-methylacetophenone; 4-methoxyacetophenone; 4-tert-butyl-2,6-dimethylacetophenone; 4-phenyl-2-butanone; 4-(4-hydroxyphenyl)-2-butanone; 1-(2-naphthalenyl)ethanone; 2-benzofuranylethanone; (3-methyl-2-benzofuranyl)ethanone; benzophenone; 1,1,2,3,3,6-hexamethyl-5-indanyl methyl ketone; 6-tert-butyl-1,1-dimethyl-4-indanyl methyl ketone; 1-[2,3-dihydro-1,1,2,6-tetramethyl-3-(1-methylethyl)-1H-5-indenyl]ethanone; 5′,6′,7′,8′-tetrahydro-3′,5′,5′,6′,8′,8′-hexamethyl-2-acetonaphthone;

aromatic and araliphatic carboxylic acids and their esters, e.g. benzoic acid; phenylacetic acid; methyl benzoate; ethyl benzoate; hexyl benzoate; benzyl benzoate; methylphenyl acetate; ethylphenyl acetate; geranylphenyl acetate; phenylethylphenyl acetate; methyl cinnamate; ethyl cinnamate; benzyl cinnamate; phenylethyl cinnamate; cinnamyl cinnamate; allyl phenoxyacetate; methyl salicylate; hexyl salicylate; cyclohexyl salicylate; cis-3-hexenyl salicylate; benzyl salicylate; phenylethyl salicylate; methyl 2,4-dihydroxy-3,6-dimethylbenzoate; ethyl 3-phenylglycidate; ethyl 3-methyl-3-phenylglycidate;

aromatic nitrogen-containing compounds, e.g. 2,4,6-trinitro-1,3-dimethyl-5-tert-butylbenzene; 3,5-dinitro-2,6-dimethyl-4-tert-butylacetophenone; cinnamic acid nitrile; 3-methyl-5-phenyl-2-pentenoic acid nitrile; 3-methyl-5-phenylpentanoic acid nitrile; methyl anthranilate; methyl N-methylanthranilate; Schiff bases of methyl anthranilate with 7-hydroxy-3,7-dimethyloctanal, 2-methyl-3-(4-tert-butylphenyl)propanal or 2,4-dimethyl-3-cyclohexenecarbaldehyde; 6-isopropylquinoline; 6-isobutylquinoline; 6-sec-butylquinoline; 2-(3-phenylpropyl)pyridine; indole; skatole; 2-methoxy-3-isopropylpyrazine; 2-isobutyl-3-methoxypyrazine;

phenols, phenyl ethers and phenyl esters, e.g. estragole; anethole; eugenyl methyl ether; isoeugenol; isoeugenyl methyl ether; thymol; carvacrol; diphenyl ether; beta-naphthyl methyl ether; beta-naphthyl ethyl ether; beta-naphthyl isobutyl ether; 1,4-dimethoxybenzene; eugenyl acetate; 2-methoxy-4-methylphenol; 2-ethoxy-5-(1-propenyl)phenol; p-cresylphenyl acetate;

heterocyclic compounds, e.g. 2,5-dimethyl-4-hydroxy-2H-furan-3-one; 2-ethyl-4-hydroxy-5-methyl-2H-furan-3-one; 3-hydroxy-2-methyl-4H-pyran-4-one; 2-ethyl-3-hydroxy-4H-pyran-4-one;

lactones, e.g. 1,4-octanolide; 3-methyl-1,4-octanolide; 1,4-nonanolide; 1,4-decanolide; 8-decen-1,4-olide; 1,4-undecanolide; 1,4-dodecanolide; 1,5-decanolide; 1,5-dodecanolide; 4-methyl-1,4-decanolide; 1,15-pentadecanolide; 1,16-hexadecanolide; 9-hexadecen-1,16-olide; 10-oxa-1,16-hexadecanolide; 11-oxa-1,16-hexadecanolide; 12-oxa-1,16-hexadecanolide; ethylene 1,12-dodecanedioate; ethylene 1,13-tridecanedioate; 2,3-dihydrocoumarin; octahydrocoumarin.

Within the framework of the uses according to the invention of the mixtures according to the invention as fragrance or flavouring mixtures (especially with an ambrette note), the present invention further relates to their use for providing (a) hair, (b) skin or (c) textile fibres with a scent, especially an ambrette scent (see above for other olfactory or gustatory notes). The present invention further relates to corresponding processes and mixtures (preferably containing surfactant). According to a related feature, the present invention further relates to the use of the mixtures according to the invention comprising compounds of formulae (I) and (II) as agents for increasing the substantivity and/or retention of a fragrance mixture, and/or as a fixative, and/or as an agent for increasing the odour of other fragrances perceived via an aqueous solution containing surfactant.

The mixtures according to the invention are also outstanding boosters (intensifiers; enhancers) for other fragrances, i.e. they intensify the sensory effect of these other fragrances. Apart from the aforementioned high stability under certain conditions of use, the additional positive secondary properties of the mixtures according to the invention also include a high strength, a good lingering power and a high substantivity.

The present invention further relates to a perfumed or flavoured product comprising

(A) a solid or semisolid carrier and a sensorially effective amount of a mixture according to the invention in contact with the solid or semisolid carrier,

or

(B) a liquid phase and, dissolved or suspended therein or diluted therewith, a sensorially effective amount of a mixture according to the invention.

The aforesaid applies to the preferred embodiment of the mixture according to the invention to be used in a perfumed or flavoured product according to the invention.

A mixture according to the invention can be prepared e.g. by preparing a premix of components (a) and (b) and mixing this premix with one or more other fragrances or flavourings (as component (c)). As a rule, components (a) and (b) are used in a total amount sufficient to create, modify and/or intensify, in the finished mixture, an olfactory or gustatory note of the radiant-ambrette, exalting, soft-woody type.

A mixture according to the invention preferably comprises a total amount of components (a) and (b) in the range from 0.00001 to 99.9 wt. %, preferably from 0.001 to 70 wt. % and particularly preferably from 0.01 to 50 wt. %, based on the total amount of fragrances or flavourings in the mixture.

If the components (a) and (b) to be used according to the invention in combination with one another are used mainly to give a fragrance or flavouring composition more radiance (emanation), rounding and/or harmony, and/or to intensify specific notes, the total amount of components (a) and (b) is preferably comparatively small and particularly preferably in the range from 0.01 to 5 wt. %, preferably in the range from 0.1 to 2 wt. %, based on the total amount of fragrances or flavourings in the mixture. If a comparatively low concentration is chosen within the preferred concentration ranges, the intrinsic olfactory or gustatory notes indicated above are not yet created in some cases, depending on the other components of the particular mixture.

The ethers of formulae (I) and (II) to be used according to the invention can be prepared by processes known per se.

Thus, for example, the alpha-ambrinol alkyl ethers of formula (I) can be prepared by alkylating alpha-ambrinol with alkyl halide, analogously to the instructions in WO 82/00030. Thus, in particular, alpha-ambrinol methyl ether (compound of formula (I) in which R=methyl) can be synthesized by reacting alpha-ambrinol with methyl iodide (cf. equation below).

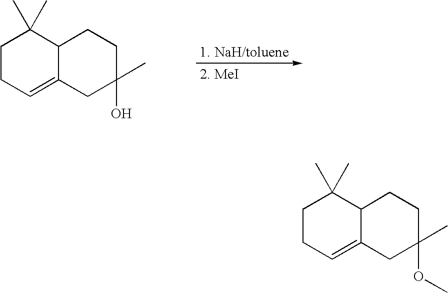

The ethers of formula (II) can be obtained e.g. from alpha-dihydroionone according to the instructions in U.S. Pat. No. 2,803,662 (reaction with alcohol in the presence of strong acid) or EP 0 330 995 (e.g. reaction with alkyl orthoformate in the presence of boron trifluoride etherate). Examples of process variants may be found by reference to said documents. An example of a reaction scheme with examples of preferred reactants is given below for the educt alpha-dihydroionone.

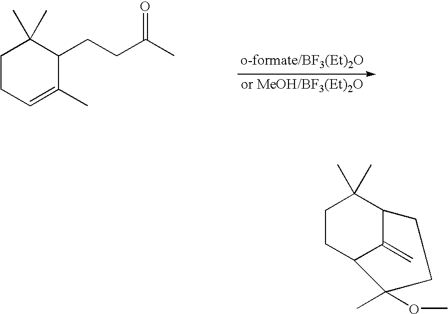

Surprisingly, the compounds of formula (I) to be used according to the invention: 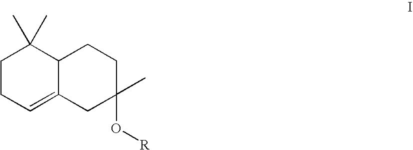
in which
R is selected from the group consisting of methyl and ethyl,
can be prepared by a process comprising the following step:
reaction of gamma-dihydroionone with
(a) an alcohol precursor, e.g. the orthoformate of the formula HC(OR)₃, in which R has the same meaning as in formula (I), or an alcohol of the formula ROH, in which R has the same meaning as in formula (I),
   in the presence of
(b) a strong acid.

The procedure is thus similar to that in documents U.S. Pat. No. 2,803,662 and EP 0 330 995, except that gamma-dihydroionone is used as the educt.

The strong acid used in the process according to the invention to prepare a compound of formula (I) is preferably a proton acid or a Lewis acid. The strong acid is preferably selected from the group consisting of hydrogen chloride gas, hydrochloric acid, sulfuric acid, phosphoric acid, trifluoroacetic acid, methanesulfonic acid, p-toluenesulfonic acid, camphorsulfonic acid, BF₃, BF₃(Et₂)O, BF₃—H₃PO₄ and acidic ion exchangers.

The compound of formula (I) prepared by the process according to the invention can then be mixed with a compound of formula (II) to prepare a mixture according to the invention. Preferably, however, simultaneously with the reaction of the gamma-dihydroionone and by reaction with the same reactants (cf. reactants (a) and (b) above), alpha-dihydroionone is converted to a compound of formula (II): 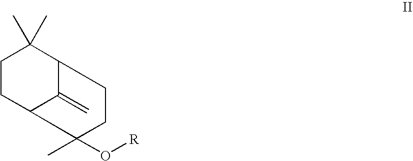
in which R has the same meaning as in formula (I).

Thus, in practice, a mixture of alpha-dihydroionone and gamma-dihydroionone is preferably subjected to the process conditions essentially described in U.S. Pat. No. 2,803,662 and EP 0 330 995. Under these process conditions the exo/endo mixture of the compounds of formula (II) is formed in known manner from the alpha-dihydroionone (the process conditions advantageously being adjusted so that the exo form predominates); the compound of formula (I) is formed simultaneously from the gamma-dihydroionone.

The product resulting from the simultaneous reaction of alpha-dihydroionone and gamma-dihydroionone is a mixture according to the invention which will hereafter be referred to as a synthetic product mixture. Preferably, a mixture according to the invention is an optionally purified synthetic product mixture comprising a total amount of at least 90 wt. %, preferably of at least 95 wt. %, of compounds of formulae (I) and (II).

It is surprising that (i) it is possible to prepare the compounds of formula (I) according to the invention from gamma-dihydroionone, (ii) the reaction can take place under process conditions essentially described in documents U.S. Pat. No. 2,803,662 and EP 0 330 995, and (iii) gamma-dihydroionone and alpha-dihydroionone can be reacted in one reaction batch and without mutual interference to give compounds of formula (I) and compounds of formula (II).

These surprising possibilities for the preparation of a mixture according to the invention comprising compounds of formulae (I) and (II) mean that it is particularly easy to prepare the mixtures according to the invention; thus the preparation according to the invention of a compound of formula (I) and a compound of formula (II) with the same meaning of R in each case, in one reaction batch, is particularly preferred according to the invention; by contrast, the separate preparation of compounds of formula (I) and compounds of formula (II) is not especially preferred since it would require an additional effort to isolate and then mix the individually prepared ethers.

Depending on the chosen reaction mixture and the actual process variant, it is possible to vary and specifically to adjust the proportions of the compounds of formula (I) and compounds of formula (II) in the synthetic product mixture. Cf. the Examples for preferred preparative processes which directly yield synthetic product mixtures comprising compounds of formula (I) as well as compounds of formula (II).

The equation below illustrates the process variants according to the invention by way of example; for the sake of simplicity, the equation relates only to compounds of formulae (I) and (II) in which R=methyl, but of course it also applies correspondingly to R ethyl:

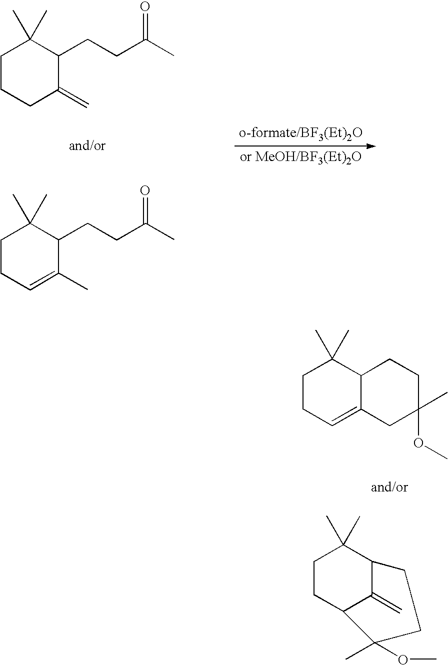

Mixtures according to the invention containing compounds of formulae (I) and (II) can be used in liquid form, either undiluted or diluted with a solvent, for perfuming or flavouring; if a solvent is present, the product according to the invention obtained comprises the solvent as a liquid phase. Examples of suitable solvents are ethanol, isopropanol, diethylene glycol monoethyl ether, glycerol, propylene glycol, 1,2-butylene glycol, dipropylene glycol, diethyl phthalate, triethyl citrate, isopropyl myristate, triacetin, etc.

Furthermore, mixtures according to the invention containing compounds of formulae (I) and (II) can be adsorbed on a carrier for ensuring both a fine distribution of the fragrances or flavourings in the product and a controlled release when used. Such carriers can be porous inorganic materials, such as light sulfate, silica gels, zeolites, gypsums, clays, clay granules, gas concrete, etc., or organic materials, such as woods, cellulose-based substances, sugars, dextrins (e.g. maltodextrin) or plastics like PVC, polyvinyl acetates or polyurethanes. If a solid or semisolid carrier is present, the perfumed or flavoured product according to the invention obtained is such that the amount of mixture according to the invention in contact with the carrier is sensorially effective.

Mixtures according to the invention can also be microencapsulated or spray-dried or take the form of inclusion complexes or extrusion products (i.e. products according to the invention) and can be added in this form e.g. to a product to be perfumed or flavoured.

Optionally, the properties of the mixtures modified in this way can be further optimized, in respect of a more specific perfume release, by coating with suitable materials; waxy plastics, e.g. polyvinyl alcohol, are preferably used for this purpose. The resulting products again constitute perfumed or flavoured products according to the invention.

Microencapsulation of the mixtures according to the invention to give products according to the invention can be effected e.g. by the so-called coacervation process with the aid of capsule materials made e.g. of polyurethane-like substances or soft gelatin. Spray-dried mixtures according to the invention can be prepared e.g. by spray drying an emulsion or dispersion containing the mixture according to the invention, it being possible for modified starches, proteins, dextrin and vegetable gums to be used as carriers. Inclusion complexes can be prepared e.g. by introducing dispersions of the fragrance or flavouring composition and cyclodextrins or urea derivatives into a suitable solvent, e.g. water. Extrusion products can be prepared by melting the mixtures according to the invention with a suitable waxy substance and by extrusion with subsequent solidification, optionally in a suitable solvent, e.g. isopropanol.

Mixtures according to the invention can be used in concentrated form, in solution or in a modified form described above for the preparation of perfumed or flavoured products according to the invention, e.g. perfume extracts, eaux de parfum, eaux de toilette, after-shave lotions, eaux de Cologne, pre-shave products, splash colognes and perfumed refreshing tissues, and for the perfuming of acidic, alkaline and neutral cleaning products, e.g. floor cleaners, window cleaners, dishwasher detergent, bath and sanitaryware cleaners, scouring cream, solid and liquid WC cleaners, carpet cleaning powders and foams, textile fresheners, ironing aids, liquid detergents, powder detergents, fabric preconditioners such as bleach, soaker and stain removers, fabric softeners, washing soaps, washing tablets, disinfectants, surface disinfectants, air purifiers in liquid or gel form or applied to a solid carrier, aerosol sprays, waxes and polishes, such as furniture polishes, floor waxes and shoe polishes, body care products, e.g. solid and liquid soaps, shower gels, shampoos, shaving soaps, shaving foams, bath oils, cosmetic emulsions of the oil-in-water, water-in-oil and water-in-oil-in-water type, such as skin creams and lotions, face creams and lotions, sun creams and lotions, after-sun creams and lotions, hand creams and lotions, foot creams and lotions, depilatory creams and lotions, after-shave creams and lotions, tanning creams and lotions, hair care products, e.g. hair sprays, hair gels, strengthening hair lotions, hair rinses, permanent and semipermanent hair colourants, hair styling products, such as cold waving and straightening products, hair tonics, hair creams and lotions, deodorants and antiperspirants, e.g. armpit sprays, roll-ons, deodorant sticks and deodorant creams, decorative cosmetic products, e.g. eye shadows, nail varnishes, make-ups, lipsticks and mascara, and candles, lamp oils, incense sticks, insecticides, repellents and propellants.

The mixtures according to the invention can be incorporated into flavoured articles or articles to be flavoured, especially preparations intended for nutrition, oral care or pleasure.

Examples of preparations intended for nutrition or pleasure are baked products (e.g. bread, dry biscuits, cakes, other baked products), sweets (e.g. chocolates, chocolate bar products, other bar products, fruit gum, hard and soft toffees, chewing gum), alcoholic or non-alcoholic drinks (e.g. coffee, tea, wine, wine-based drinks, beer, beer-based drinks, liqueurs, schnapps, brandies, fruit-based soft drinks, isotonic drinks, refreshing drinks, nectars, fruit and vegetable juices, fruit or vegetable juice preparations), instant drinks (e.g. instant cocoa drinks, instant tea drinks, instant coffee drinks), meat products (e.g. ham, fresh or uncooked sausage preparations, spiced or marinated fresh or salted meat products), eggs or egg products (dried egg, egg white, egg yolk), cereal products (e.g. breakfast cereals, muesli bars, precooked ready-to-eat rice products), milk products (e.g. milk drinks, ice milk, yoghurt, kefir, fresh cheese, soft cheese, hard cheese, dried milk powder, whey, butter, buttermilk, partially or fully hydrolyzed products containing milk protein), products from soya protein or other soya bean fractions (e.g. soya milk and products manufactured therefrom, preparations containing soy a lecithin, fermented products such as tofu or tempe, products manufactured therefrom, soya sauces), fruit preparations (e.g. jams, fruit sorbets, fruit sauces, fruit fillings), vegetable preparations (e.g. ketchup, sauces, dried vegetables, frozen vegetables, precooked vegetables, vegetables pickled in vinegar, preserved vegetables), snacks (e.g. baked or fried potato chips or potato pastry products, bread dough products, extrudates based on maize or peanuts), fat-based and oil-based products or emulsions thereof (e.g. mayonnaise, remoulade, dressings, spice preparations), other ready meals and soups (e.g. dried soups, instant soups, precooked soups), spices, spice mixtures and especially seasonings used e.g. in the snack sector. After incorporation of the mixtures according to the invention, these preparations are preparations according to the invention (as an example of flavoured products according to the invention).

Preparations according to the invention can take the form of semiprepared products or spice mixtures.

Preparations according to the invention can be used especially as semiprepared products for the manufacture of other preparations intended for nutrition or pleasure, especially in spray-dried form. Preparations according to the invention can also take the form of capsules, tablets (uncoated or coated tablets, e.g. enteric-coated tablets), dragees, granules, pellets, solid mixtures, dispersions in liquid phases, emulsions, powders, solutions, pastes or other preparations as food supplements which can be swallowed or chewed.

Preparations according to the invention intended for oral care are especially oral and/or dental care products, such as toothpastes, tooth gels, tooth powders, mouthwashes, chewing gums and other oral care products.

Other conventional active ingredients, bases, auxiliary substances and additives for preparations according to the invention intended for nutrition, oral care or pleasure can be present in amounts of 5 to 99.999999 wt. %, preferably of 10 to 80 wt. %, based on the total weight of the preparation. The preparations can also contain water in an amount of up to 99.999999 wt. %, preferably of 5 to 80 wt. %, based on the total weight of the preparation.

In one preferred embodiment, preparations according to the invention containing compounds of formulae (I) and (II) (as an example of flavoured products according to the invention) are prepared by incorporating a mixture according to the invention, as such, as a solution (e.g. in ethanol, water or 1,2-propylene glycol) or in the form of a mixture with a solid or liquid carrier (e.g. maltodextrin, starch, silica gel), other flavourings and optionally other auxiliary substances and/or stabilizers (e.g. natural or synthetic polysaccharides and/or vegetable gums, such as modified starches or gum arabic), into a base preparation intended for nutrition, oral care or pleasure. Advantageously, preparations according to the invention in the form of a solution and/or suspension or emulsion can also be converted to a solid preparation according to the invention (semiprepared product) by spray drying.

As semiprepared products, the spray-dried solid preparations according to the invention (as an example of articles according to the invention) are particularly suitable for the manufacture of other preparations according to the invention. The spray-dried solid preparations according to the invention preferably contain 50 to 95 wt. % of carriers, especially maltodextrin and/or starch, and 5 to 40% of auxiliary substances, preferably natural or synthetic polysaccharides and/or vegetable gums, such as modified starches or gum arabic.

In another preferred embodiment for the manufacture of preparations according to the invention, firstly a mixture according to the invention and optionally other constituents of the preparation according to the invention are incorporated into emulsions, liposomes, e.g. based on phosphatidylcholine, microspheres, nanospheres or else capsules, granules or extrudates made of a matrix suitable for ordinary and semi-luxury foods, e.g. starch, starch derivatives (e.g. modified starch), cellulose or cellulose derivatives (e.g. hydroxypropyl cellulose), other polysaccharides (e.g. dextrin, alginate, curdlan, carrageenan, chitin, chitosan, pullulan), natural fats or natural waxes (e.g. beeswax, carnauba wax), or proteins, e.g. gelatin or other natural products (e.g. shellac). Here, depending on the matrix, the products can be obtained by spray drying, spray granulation, melt granulation, coacervation, coagulation, extrusion, melt extrusion, emulsion processes, coating or other suitable encapsulation processes, or optionally a suitable combination of the aforesaid processes. In another preferred preparative process for a preparation according to the invention, firstly a mixture according to the invention is complexed with one or more complexing agents, e.g. cyclodextrins or cyclodextrin derivatives, preferably alpha- or gamma-cyclodextrin, and used in this complexed form.

A particularly preferred preparation according to the invention is one in which the matrix is chosen so that the mixture according to the invention of compounds of formulae (I) and (II) is released from the matrix in a delayed manner, thereby achieving a long-lasting effect. A fat, wax, polysaccharide or protein matrix is particularly preferred in this respect.

As other constituents for preparations according to the invention intended for nutrition or pleasure, it is possible to use conventional bases, auxiliary substances and additives for ordinary or semi-luxury foods, e.g. water, mixtures of fresh or processed, vegetable or animal bases or raw materials (e.g. raw, roasted, dried, fermented, smoked and/or boiled meat, bone, gristle, fish, vegetables, fruits, herbs, nuts, vegetable or frjit juices or pastes, or mixtures thereof), digestible or indigestible carbohydrates (e.g. sucrose, maltose, fructose, glucose, dextrins, amylose, amylopectin, inulin, xylan, cellulose, tagatose), sugar alcohols (e.g. sorbitol, erythritol), natural or hydrogenated fats (e.g. tallow, lard, palm butter, coconut butter, hydrogenated vegetable fat), oils (e.g. sunflower oil, peanut oil, maize oil, olive oil, fish oil, soya oil, sesame oil), fatty acids or their salts (e.g. potassium stearate), proteogenic or non-proteogenic amino acids and related compounds (e.g. γ-aminobutyric acid, taurine), peptides (e.g. glutathione), native or processed proteins (e.g. gelatin), enzymes (e.g. peptidases), nucleic acids, nucleotides, taste correctors for unpleasant gustatory impressions, other taste modulators for other gustatory impressions that are not normally unpleasant, other taste modulating substances (e.g. inositol phosphate, nucleotides such as guanosine monophosphate or adenosine monophosphate, or other substances such as sodium glutamate or 2-phenoxypropionic acid), emulsifiers (e.g. lecithins, diacylglycerols, gum arabic), stabilizers (e.g. carrageenan, alginate), preservatives (e.g. benzoic acid, sorbic acid), antioxidants (e.g. tocopherol, ascorbic acid), chelators (e.g. citric acid), organic or inorganic souring agents (e.g. malic acid, acetic acid, citric acid, tartaric acid, phosphoric acid), bitter substances (e.g. quinine, caffeine, limonin, amarogentin, humulones, lupolones, catechols, tannins), mineral salts (e.g. sodium chloride, potassium chloride, magnesium chloride, sodium phosphates), substances for inhibiting enzymatic browning (e.g. sulfite, ascorbic acid), ethereal oils, plant extracts, natural or synthetic dyes or coloured pigments (e.g. carotinoids, flavonoids, anthocyans, chlorophyll and derivatives thereof), spices, trigeminally active substances or plant extracts containing such trigeminally active substances, synthetic, natural or nature-identical flavourings or fragrances, and odour correctors.

Dental care products (as an example of preparations intended for oral care) containing a mixture according to the invention generally comprise an abrasive system (abrasive powder or polishing agent), e.g. silicic acids, calcium carbonates, calcium phosphates, aluminium oxides and/or hydroxyapatites, surface-active substances, e.g. sodium laurylsulfate, sodium laurylsarcosinate and/or cocamidopropylbetaine, humectants, e.g. glycerol and/or sorbitol, thickeners, e.g. carboxymethyl cellulose, polyethylene glycols, carrageenan and/or Laponite®, sweeteners, e.g. saccharin, taste correctors for unpleasant gustatory impressions, taste correctors for other gustatory impressions that are not normally unpleasant, taste modulating substances (e.g. inositol phosphate, nucleotides such as guanosine monophosphate or adenosine monophosphate, or other substances such as sodium glutamate or 2-phenoxypropionic acid), cooling agents, e.g. menthol, menthol derivatives (e.g. L-menthol, L-menthyl lactate, L-menthyl alkyl carbonates, menthone ketals, menthanecarboxylic acid amides), 2,2,2-trialkylacetic acid amides (e.g. 2,2-diisopropylpropionic acid methylamide), icilin and icilin derivatives, stabilizers and active ingredients, e.g. sodium fluoride, sodium monofluorophosphate, tin difluoride, quaternary ammonium fluorides, zinc citrate, zinc sulfate, tin pyrophosphate, tin dichloride, mixtures of different pyrophosphates, triclosan, cetylpyridinium chloride, aluminium lactate, potassium citrate, potassium nitrate, potassium chloride, strontium chloride, hydrogen peroxide, flavourings and/or sodium bicarbonate, or odour correctors.

Chewing gums (as another example of preparations intended for oral care) containing a mixture according to the invention generally comprise a chewing gum base, i.e. a compound that becomes plastic on chewing, various types of sugars, sugar substitutes, other sweet-tasting substances, sugar alcohols, taste correctors for unpleasant gustatory impressions, other taste modulators for other gustatory impressions that are not normally unpleasant, taste modulating substances (e.g. inositol phosphate, nucleotides such as guanosine monophosphate or adenosine monophosphate, or other substances such a sodium glutamate or 2-phenoxypropionic acid), humectants, thickeners, emulsifiers, flavourings and stabilizers, or odour correctors.

Apart from the mixture according to the invention, the preparations according to the invention can preferably also contain a flavouring composition in order to round and refine the taste and/or odour of the preparation. Suitable (additional) flavouring compositions contain e.g. synthetic, natural or nature-identical flavourings, fragrances and taste enhancers, as well as suitable auxiliary substances and carriers.

The invention is illustrated in greater detail below with the aid of Examples. Unless indicated otherwise, all the data are by weight.

### Example 1

### Preparation of a Mixture of Methyl Ethers of Formulae (I) and (II) (Based on the Process According to U.S. Pat. No. 2,803,662)

300 g of boron trifluoride/methanol complex were placed in a 2 l stirred vessel and 291.4 g (1.5 mol) of a mixture of gamma-dihydroionone (55%) and alpha-dihydroionone (40%) were added dropwise at 10° C. The resulting mixture was then stirred for 15 hours at RT. The reaction solution was then cautiously decomposed with 300 ml of 10% NaOH, the aqueous phase was separated off and the organic phase was washed with water until the washings were neutral, and concentrated. The crude product (312 g) was fractionated under vacuum on a 30 cm Vigreux column.

Yield: 234 g (75% of theory) B.p.: 130°-140° C./8 mbar

GC evaluation (20 m ZB-WAX, internal diameter 0.18 μm/60-9-220° C. cold application system):

| Index | Product | Wt. % |
|---|---|---|
| 1601 | endo-methoxy ether of | 13.4% |
| | formula II | |
| 1631 | exo-methoxy ether of | 28.7% |
| | formula II | |
| 1669 | alpha-ambrinol methyl | 53.9% |
| | ether | |

### Example 2

### Preparation of a Mixture of Methyl Ethers of Formulae (I) and (II) (on the Basis of the Process According to EP 0 330 995)

291.4 g (1.5 mol) of a mixture of gamma-dihydroionone (55%) and alpha-dihydroionone (40%), and 175 g (1.66 mol) of trimethyl orthoformate in 450 ml of methylene chloride, were placed in a 2 l stirred vessel. A solution of 12 g of boron trifluoride etherate in 150 ml of methylene chloride was then added over one hour, with stirring and cooling. The mixture was then stirred for 1.5 hours under reflux. After cooling to room temperature, the reaction solution was cautiously decomposed with 300 ml of 10% NaOH, the aqueous phase was separated off and the organic phase was washed with water until the washings were neutral, and concentrated. The crude product (315 g) was fractionated under vacuum on a 30 cm Vigreux column.

Yield: 443.5 g (92% of theory) B.p.: 130°-140° C./8 mbar

GC evaluation (20 m ZB-WAX, internal diameter 0.18 μm/60-9-220° C. cold application system):

| Index | Product | Wt. % |
|---|---|---|
| 1601 | endo-methoxy ether of | 9.6% |
| | formula II | |
| 1631 | exo-methoxy ether of | 32.3% |
| | formula II | |
| 1669 | alpha-ambrinol methyl | 55.9% |
| | ether | |

GC/MS Data for the Methyl Ethers
1. endo-2-methoxy-9-methylene-2,6,6-trimethylbicyclo(3.3.1)nonane

MS: m/z (%)=176 (40, M⁺−32), 161 (37), 133 (19), 120 (16), 105 (16), 85 (100), 55 (18), 43 (9).

2. exo-2-methoxy-9-methylene-2,6,6-trimethylbicyclo(3.3.1)nonane

MS: m/z (%)=176 (44, M⁺−32), 161 (38), 133 (20), 120 (18), 105 (17), 91(10), 85 (100), 55 (18).

3. alpha-ambrinol methyl ether

MS: m/z (%)=208 (5, M⁺), 176 (100), 161 (84), 133 (24), 120 (23), 106 (31), 105 (39), 91 (22), 85 (84).

GC/MS for the Ethyl Ethers

1. exo-2-ethoxy-9-methylene-2,6,6-trimethylbicyclo(3.3.1)nonane

MS: m/z (%)=222 (0.2 M⁺), 176 (44), 161 (36), 133 (17), 105 (15), 99 (100), 91 (16), 71 (58), 43 (15).

2. endo-2-ethoxy-9-methylene-2,6,6-trimethylbicyclo(3.3.1)nonane

MS: m/z (%)=222 (0.1 M⁺), 176 (38), 161 (31), 133 (15), 105 (13), 99 (100), 91 (14), 71 (57), 43 (16).

3. alpha-ambrinol ethyl ether

MS: m/z (%)=222 (0.4 M⁺), 176 (62), 161 (42), 133 (17), 120 (16), 105 (19), 99 (100), 91 (15), 71 (47).

### Example 3

### Perfume Composition

### Fragrance Composition

| | |
|---|---|
| Agrumex LC | 10.00 |
| Amarocit ® 10% in DPG | 10.00 |
| Ambroxide cryst. | 10.00 |
| Basil oil | 10.00 |
| Calone 1951 10% in DPG | 10.00 |
| Cedarwood oil | 10.00 |
| Cedrol cryst. | 50.00 |
| Citral 10% in DPG | 10.00 |
| Citronellol | 5.00 |
| Coumarin | 10.00 |
| Cyclogalbanat ® 10% in DPG | 15.00 |
| Dihydromyrcenol | 80.00 |
| Farenal ® 10% in DPG | 5.00 |
| Galbex 10% in DPG | 25.00 |
| Globalide ® | 80.00 |
| Globanone ® | 40.00 |
| Hedione | 90.00 |
| Helional | 20.00 |
| Heliotropin | 5.00 |
| Hexenol cis-3 10% in DPG | 15.00 |
| Hexenyl salicylate cis-3 | 10.00 |
| Beta-ionone | 5.00 |
| Iso E Super | 180.00 |
| Isodamascon ® 10% in DPG | 10.00 |
| Isomuscone (cyclohexadecanone) | 20.00 |
| Isoraldein 70 | 20.00 |
| Ketamber 10% in TEC | 25.00 |
| Lavandin oil Grosso Nat. | 15.00 |
| Lilial | 20.00 |
| Linalool | 20.00 |
| Linalyl acetate | 40.00 |
| Mandarin oil brasil. green | 50.00 |
| Timberol ® | 40.00 |
| Vanillin | 5.00 |
| Veloutone 10% in DPG | 20.00 |
| Ysamber K ® | 10.00 |
| Total | 1000.00 |

DPG: dipropylene glycol, TEC: triethyl citrate

Odour description of the perfume composition without the addition of a mixture according to the invention: fresh, woody.

In the perfumers' opinion, the addition of 3 wt. % of a mixture according to the invention from Example 2 makes this perfume composition fresher, more radiant, more rounded and more harmonious, a sweet, ambrette note being added and the woody and flowery aspects being intensified. Through its intrinsic odour and its modifying and intensifying effect (booster effect), the mixture according to the invention from Example 2 which is used gives the composition its own character and combines the different olfactory elements.

### Example 4

### Perfume Composition

### Fragrance Composition

| | |
|---|---|
| Allylcyclohexyl propionate | 3.00 |
| Amyl salicylate | 2.00 |
| Benzyl acetate | 64.00 |
| Citronellol | 122.00 |
| Citral 10% in DPG | 2.00 |
| Cyclamen aldehyde | 10.00 |
| Dihydromyrcenol | 3.00 |
| Dimethylbenzylcarbinyl acetate | 3.00 |
| Ethyl salicylate 10% in DPG | 2.00 |
| Eugenol | 3.00 |
| Indoflor 10% in DPG | 16.00 |
| Galaxolide 50% in DPG | 164.00 |
| Geraniol | 35.00 |
| Dihydromethyl jasmonate | 6.00 |
| Heliotropin | 4.00 |
| Hexylcinnamaldehyde | 121.00 |
| Vertocitral | 4.00 |
| Hydroxycitronellal | 42.00 |
| Indole | 2.00 |
| Isobutyl salicylate | 6.00 |
| Lavandin oil Grosso Nat. | 6.00 |
| Acetylcedrene | 10.00 |
| Lilial | 190.00 |
| Linalool | 35.00 |
| Linalyl acetate | 10.00 |
| Methyl anthranilate 10% in DPG | 5.00 |
| Nerol | 10.00 |
| Orange oil | 6.00 |
| Phantolide | 4.00 |
| Phenylacetaldehyde dimethyl acetal | 6.00 |
| Phenylethyl alcohol | 75.00 |
| Rosatol 10% in DPG | 6.00 |
| Sandalwood oil | 3.00 |
| Sandranol | 16.00 |
| Trifernal | 2.00 |
| Tonalide | 2.00 |
| Total | 1000.00 |

DPG: dipropylene glycol

Odour description of the perfume composition without the addition of a mixture according to the invention: flowery, lily of the valley.

In the perfumers' opinion, the addition of 6 wt. % of a mixture according to the invention from Example 1 gives this perfume composition a new lease of life. The flowery impression is appreciably intensified. The composition has a more radiant, more rounded and more harmonious effect, a natural, ambrette note being added. Through its intrinsic odour and its modifying and intensifying effect (booster effect), the mixture according to the invention used gives the composition its own character and combines the different olfactory elements.

### Example 5

### Shampoo

A mixture according to the invention from Example 1 was incorporated in a dose of 0.5 wt. % into a shampoo base of the following composition:

| | |
|---|---|
| Sodium laurylethersulfate | 12% |
| (e.g. Texapon NSO, Cognis Deutschland GmbH) | |
| Cocamidopropylbetaine | 2% |
| (e.g. Dehyton K, Cognis Deutschland GmbH) | |
| Sodium chloride | 1.4% |
| Citric acid | 1.3% |
| Phenoxyethanol, methyl-, ethyl-, butyl- | 0.5% |
| and propylparaben | |
| Water | 82.8% |

The pH of the shampoo base was about 6. It was used to prepare 100 ml of a 20 wt. % aqueous shampoo solution. Two swatches of hair were washed together for 2 minutes in this shampoo solution and then rinsed for 20 seconds under lukewarm running water.

One swatch was packed wet in aluminium foil and the other was dried with a hairdryer. Both swatches were assessed for odour by a panel.

Odour description in both cases: very strongly radiant, ambrette, exalting, soft-woody, reminiscent of ambergris and civet.

### Example 6

### Fabric Softener

The perfume composition from Example 3 (after the addition of 3 wt. % of a mixture according to the invention from Example 2) was incorporated in a dose of 0.5 wt. % into a fabric softener base of the following composition:

| | |
|---|---|
| Quaternary ammonium methosulfate (Esterquat), | 5.5% |
| ca. 90% | |
| (e.g. Rewoquat WE 18, Witco Surfactants GmbH) | |
| Alkyldimethylbenzylammonium chloride, | 0.2% |
| ca. 50% | |
| (e.g. Preventol R50, Bayer AG) | |
| Dye solution, ca. 1% | 0.3% |
| Water | 94.0% |

The pH of the fabric softener base was in the range from 2 to 3. Two cloths were rinsed in a Linetest machine on the soft rinse programme for 30 minutes at 20° C. with 370 g of a 1% aqueous fabric softener solution based on the fabric softener base comprising 0.5 wt. % of the mixture according to the invention. The cloths were wrung out and then spun for 20 seconds. One cloth was heat-sealed wet and the other was hung out to dry. Both cloths were then assessed for odour by a panel.

Odour description in both cases: flowery, woody, fresh, radiant, ambrette and woody aspects with light, sweet undertones, rounded and harmonious olfactory impression.

### Example 7

### Washing Powder

The perfume oil composition from Example 4 (after the addition of 6 wt. % of a mixture according to the invention from Example 1) was incorporated in a dose of 0.4 wt. % into a washing powder base of the following formulation:

| | |
|---|---|
| Linear Na alkylbenzenesulfonate | 8.8% |
| Ethoxylated fatty alcohol C12-18 (7 EO) | 4.7% |
| Na soap | 3.2% |
| DOW CORNING(R) 2-4248S | 3.9% |
| POWDERED ANTIFOAM, | |
| silicone oil on zeolite as carrier | |
| Zeolite 4A | 28.3% |
| Na carbonate | 11.6% |
| Na salt of an acrylic acid/maleic acid | 2.4% |
| copolymer (Sokalan CP5) | |
| Na silicate | 3.0% |
| Carboxymethyl cellulose | 1.2% |
| Dequest 2066 | 2.8% |
| ([[(phosphonomethyl)imino]bis[(ethylenenitrilo)bis- | |
| (methylene)]]tetrakisphosphonic acid, sodium salt) | |
| Optical brightener | 0.2% |
| Na sulfate | 6.5% |
| Protease | 0.4% |
| Sodium perborate tetrahydrate | 22.0% |
| TAED | 1.0% |

Two cloths were washed in a Linetest machine on the main wash programme for 45 minutes at 60° C. with 370 g of a 1% aqueous washing powder solution based on the washing powder base comprising 0.4 wt. % of the perfume oil composition from Example 4 (the pH of the washing powder solution is well in the basic range). The cloths were first rinsed for 5 minutes with cold water, wrung out and then spun for 20 seconds. One cloth was heat-sealed wet and the other was hung out to dry. Both cloths were then assessed for odour by a panel.

Odour description in both cases: strongly flowery, radiant, natural, ambrette note with light, sweet and woody undertones, rounded and harmonious olfactory impression.

1. A mixture comprising:
at least one compound having formula (I); 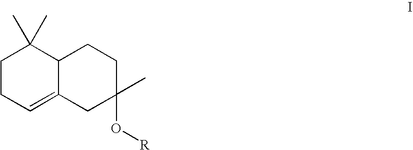
and
at least one compound having formula (II), 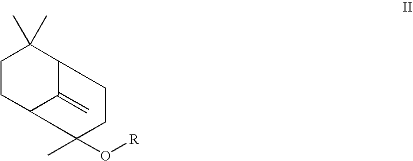
whereing
R has the same meaning or a different meaning in formula (I) and formula (II) and is either methyl or ethyl.

2. The mixture according to claim 1, wherein the mixture includes either:
(a) one compound of formula (I) and one compound of formula (II) where R is the same; or
(b) a second compound having formula (I) where R is different from the first compound having formula (I); or
(c) a second compound having formula (II) where R is different from the first compound having formula (II).

3. The mixture according to claim 2 wherein for (a) R is methyl.

4. The mixture according to claim 1, wherein a total amount of compound having formula (II) in the mixture is chosen so that an animal olfactory note otherwise caused by the compound having formula (I) is effectively masked.

5. The mixture according to claim 1, wherein a ratio of a total amount of compounds having formula (I) to a total amount of compounds having formula (II) ranges from 100:1 to 1:10, preferably from 3:1 to 1:3 and most preferably from 2:1 to 1:1.

6. The mixture according to claim 1, wherein the mixture is an optionally purified synthetic product mixture comprising a total amount of at least 90 wt. %, preferably of at least 95 wt. %, of compounds having formula (I) and (II).

7. The mixture according to claim 1, wherein the mixture has at least one of the following olfactory properties: very strongly radiant, ambrette, exalting, soft-woody, reminiscent of ambergris and civet, very natural olfactory impression.

8. The mixture according to claim 1, further comprising at least one fragrance or flavouring, said fragrance or flavouring having an olfactory property that is either woody or flowery.

9. The use of a mixture according to claims 1 as a fragrance or flavouring mixture.

10. The use of a mixture according to claim 4 as a fragrance or flavouring mixture.

11. The method of using a mixture comprising a compound having a formula (II), 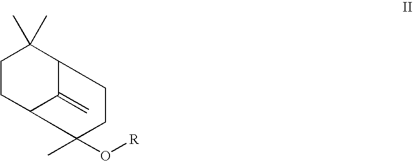
as an agent for masking an unpleasant olfactory note, wherein R is either methyl or ethyl.

12. The method according to claim 11, wherein the mixture further comprises a compound having a formula (I), 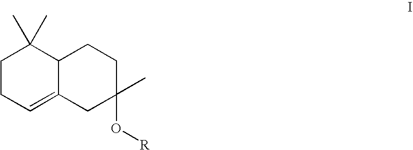
wherein
R is either methyl or ethyl, and
has the same meaning or a different meaning in formula (I) and formula (II).

13. The method of using a compound having formula (I), 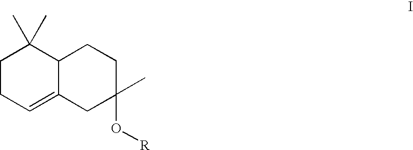
for modifying olfactory properties of a compound of formula (II), 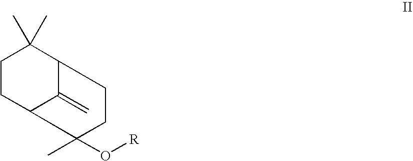
wherein
R is either methyl or ethyl, and has the same meaning or a different meaning in formula (I) and formula (II),
thereby making it more radiant with a stronger nuance of ambrette.

14. A perfumed or flavoured product comprising:
(A) a solid or semisolid carrier;
and
a sensorially effective amount of a mixture in contact with the solid or semisolid carrier;
or
(B) a liquid phase;
and,
dissolved or suspended therein or diluted therewith, a sensorially effective amount of a mixture, wherein the mixture is one according to claim 1.

15. The perfumed product according to claim 14, wherein the perfumed product is either perfume extracts, eaux de parfum, eaux de toilette, after-shave lotions, eaux de Cologne, pre-shave products, splash colognes, perfumed refreshing tissues, acidic, alkaline and neutral cleaning products, textile fresheners, ironing aids, liquid detergents, powder detergents, fabric preconditioners, fabric softeners, washing soaps, washing tablets, disinfectants, surface disinfectants, air purifiers, aerosol sprays, waxes and polishes, body care products, hand creams and lotions, foot creams and lotions, depilatory creams and lotions, after-shave creams and lotions, tanning creams and lotions, hair care products, deodorants and antiperspirants, decorative cosmetic products, candles, lamp oils, incense sticks, insecticides, repellents or propellants.

16. A method for creating, modifying and/or intensifying an odour, comprising the following step:
bringing a product into contact, or mixing it, with a sensorially effective amount of a mixture according to claims 1.

17. A method for the preparation of a compound having formula (I): 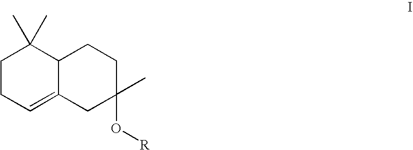
wherein
R is either methyl or ethyl,
comprising the following step:
reacting a gamma-dihydroionone with an alcohol precursor having a formula HC(OR)₃;
or
an alcohol of the formula ROH, wherein,
R has the same meaning as in formula (I), in the presence of a strong acid.

18. The method according to claim 17 wherein the strong acid is a proton acid or a Lewis acid.

19. The method according to claim 17 wherein the strong acid is either hydrogen chloride gas, hydrochloric acid, sulfuric acid, phosphoric acid, trifluoroacetic acid, methanesulfonic acid, p-toluenesulfonic acid, camphorsulfonic acid, BF₃, BF₃(Et₂)O, BF₃—H₃PO₄ or acidic ion exchangers.

20. The method according to claim 17 wherein, simultaneously with the reaction of the gamma-dihydroionone and by reaction with the same reactants, alpha-dihydro-ionone is converted to a compound of formula (II), 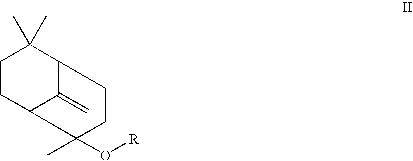
wherein R has the same meaning as in formula (I).
